(19) United States
(12) Patent Application Publication
(10) Pub. No.: US 2009/0311216 A1
(43) Pub. Date: Dec. 17, 2009

(54) RECOMBINANT INTERFERON-BETA WITH ENHANCED BIOLOGICAL ACTIVITY

(76) Inventors: Deborah Johnson-Jackson, Canby, CA (US); Kenji Furuya, Bourlder, CO (US); Isabel Zaror, Berkeley, CA (US); Douglas Larson, Walnut Creek, CO (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 12/375,958

(22) PCT Filed: Jul. 24, 2007
(86) PCT No.: PCT/US07/16722

§ 371 Date: Apr. 13, 2009

Related U.S. Application Data
(60) Provisional application No. 60/836,541, filed on Aug. 8, 2006.

(51) Int. Cl. A61K 38/21; C07K 14/565; C12P 21/02; C12Q 1/37; C12N 15/22; C12N 1/21; C12N 5/10; A61P 37/00

(52) U.S. Cl. 424/85.6; 530/351; 435/69.51; 435/23; 435/252.3; 536/23.52; 435/252.33; 435/348; 435/358

(57) ABSTRACT

Human interferon-β protein analogs in which the asparagine at position 25, numbered in accordance with native human interferon-β, is recombinantly replaced with an aspartate residue exhibit a biological activity of human interferon-β (e.g. IFN-β 1b) at an increased level relative to IFN-β 1b. These analogs are obtained by introducing a gene coding for Asp25 IFN-β into a cell and expressing the recombinant protein. The resulting IFN-β protein analog is suitable for large scale manufacturing for incorporation in HA-containing or HA-free therapeutics for treatment of diseases including multiple sclerosis. A reduced Lys endoproteinase-C peptide map technique that produces a fingerprint profile for proteins using an enzymatic digest followed by RP-HPLC is also useful in quality control as an ID test for the IFN-β protein analog products.

### BACKGROUND

1. Technical Field

This invention is in the general area of biologically active protein chemistry. More specifically it relates to mutationally altered interferon-β analogs that differ from the native protein by substitutions, deletions or modifications of cysteine, asparagine and other residues.

2. Background Art

Interferon-β has been found to be useful in the treatment of human disease, in particular multiple sclerosis. Multiple sclerosis (MS) is a chronic, often disabling disease of the central nervous system that occurs when a protective sheath surrounding nerve fibers breaks down. About thirty percent of MS patients suffer from a relapsing-remitting form of the disease in which symptoms disappear totally or partially after a flare-up and are followed by a period of stability that can last for months or years. Administration of beta interferon (Interferon-β or IFN-β) has been approved by FDA for treatment of relapsing-remitting forms of MS. Currently, the three interferon-β products marketed for MS therapy, Betaseron®, Avonex®, and Rebif®, have combined sales exceeding three billion dollars a year. There is a continuous need for more effective IFN-β products and for more efficient methods of making them.

Recombinant DNA (rDNA) techniques have been developed to facilitate the large scale manufacturing of Interferon-β based pharmaceuticals. Recombinant DNA molecules are DNA molecules constructed outside of living cells by joining natural or synthetic DNA segments to DNA molecules that can replicate in a living cell, or molecules that result from their replication. Recombinant DNA techniques allow large-scale production of native or mutationally altered proteins. One problem in particular that needed to be addressed by these techniques was that human beta interferon, the amino acid sequence of which is provided in FIG. 1 (SEQ ID NO: 1), contains cysteine residues at positions 17, 31, and 141, (Gene (1980) 10:11-15 and Nature (1980) 285:542-547), which are capable of forming intermolecular or intramolecular links during some of the production steps. These links may form during renaturation of denatured IFN-β leading to undesired misfolded structures and aggregates. In the course of the microbial preparation of IFN-β by rDNA techniques, it has been observed that dimers and oligomers of IFN-β are formed in extracts containing high concentrations of IFN-β due to this intermolecular linking. This multimer formation renders purification and isolation of IFN-β very laborious and time-consuming and necessitates several additional steps in purification and isolation procedures such as reducing the protein during purification and reoxidizing it to restore it to its original conformation, thereby increasing the possibility of incorrect disulfide bond formation. In addition, this multimer formation has been associated with low specific biological activity.

In order to address these issues, refined rDNA techniques have been developed to alter microbially produced biologically active IFN-β protein analogs in a manner that does not affect their activity adversely, but reduces or eliminates their ability to form intermolecular crosslinks or intramolecular bonds that cause the protein to adopt an undesirable tertiary structure (e.g., a conformation that reduces the activity of the protein). Directed mutagenesis techniques have been successfully used to form mutationally altered biologically active protein analogs (a “protein analog” refers herein to a synthetic protein in which one or more amino acids has been genetically and/or chemically and/or thermally modified and that retains a biological activity of the parent protein) that retain a desired activity of their parent proteins but lack the ability to form intermolecular links or undesirable intramolecular disulfide bonds. Synthetic protein analogs of IFN-β biologically active protein which have the cysteine residue at position 17 deleted or replaced by another amino acid have been found to have the desired activity and characteristics.

In particular, Interferon-β 1b (IFN-β 1b), a synthetic, recombinant protein analog of IFN-β, is a biologically active protein which has the cysteine residue at position 17 replaced by a serine residue. As a microbially produced protein, IFN-β 1b is unglycosylated. It also has an N-terminal methionine deletion. IFN-β 1b has been formulated into a successful pharmaceutical marketed as Betaseron® that has been shown to be effective for treatment and management of MS. This protein analog, materials and techniques for its manufacture, its formulation as a therapeutic and its use to treat MS are described and claimed in a number of US patents and applications including U.S. Pat. No. 4,588,585, issued May 13, 1986; U.S. Pat. No. 4,737,462, issued Apr. 12, 1988; and U.S. Pat. No. 4,959,314, issued Sep. 25, 1990; each of which is incorporated by reference herein for their disclosure of these features.

Large scale manufacturing of IFN-β for pharmaceuticals is also conducted from mammalian sources, in particular Chinese hamster ovary (CHO) cells. This IFN-β analog, referred to as IFN-β 1a, lacks the Ser17 mutation of IFN-β 1b and is glycosylated. IFN-β 1a is formulated into therapeutic products marketed as Avonex® and Rebif®.

As with most therapeutics, there is a continual desire to identify and manufacture more potent biologically active agents. In the case of IFN-β based pharmaceuticals, an IFN-β analog with increased biological activity would be desirable.

In addition, some IFN-β pharmaceutical formulations, including Betaseron®, contain human albumin (HA or HSA), a common protein stabilizer. HA is a human blood product and is in increasingly low supply. Accordingly, more recently there has been a desire for HA-free drug formulations, and a stable and effective HA-free IFN-β formulation would be desirable.

In general, IFN-β compositions having increased biological activity, which can be formulated with or without HA and methods of making such compositions are needed.

### SUMMARY OF THE INVENTION

The present invention addresses these needs by providing a purified and isolated recombinant human interferon-β protein analog containing IFN-β in which the asparagine at position 25, numbered in accordance with native interferon-β, has been replaced by an aspartate residue via a mutation. The Asn25Asp mutation is introduced using rDNA techniques, by providing a gene coding for Asp25 IFN-β, and by introducing this gene into the cells, such as microbial cells, or eukaryotic cells (e.g. CHO cells or insect cells), to effect the expression of the Asp25 IFN-β protein analog in these cells. In some embodiments, rDNA used for transformation of the cells also includes a promoter sequence. The recombinant interferon-β protein analog is free from the native Asn25 IFN-β and exhibits biological activity of IFN-β (e.g. IFN-β 1b) at an increased level relative to IFN-β 1b. This protein analog is stable in the absence of HA and can be formulated into HA-free or HA-containing therapeutic compositions.

The recombinant interferon-β protein analog, in addition to Asp25 IFN-β protein analog may also contain its degradation products, such as Isoasp25 and Imide25 protein variants shown in FIG. 2.

In a specific embodiment, the recombinant Asp25 IFN-β is a synthetic human interferon-β 1b protein analog in which cysteine at position 17, numbered in accordance with native interferon-β, is deleted or replaced by a neutral amino acid, in particular serine, and the asparagine at position 25, is recombinantly exchanged for aspartate. In this embodiment the Asp25 IFN-β has an N-terminal methionine deletion and is unglycosylated. Specifically, the Asp25 IFN-β may have a primary amino acid sequence as set forth in FIG. 3 (SEQ ID NO: 2).

The human interferon-β protein analog can be characterized using a reduced Lys-C endoproteinase assay performed at a pH of at least about 6.5, preferably in the range from about 6.9 to about 7.1, and most preferably at pH of about 7. Lys-C digest performed at this pH range cleaves the protein at specific sites without altering the relative amounts of decomposition products in the sample. The peptide fragments obtained in the digest can be further separated by reverse phase (RP) HPLC and identified by Edman sequencing.

Formulations of the active protein analog compounds in therapeutic compositions, which include HA-free and HA-containing formulations, and methods of making and use are also provided.

These and other objects and features of the invention will become more fully apparent when the following detailed description of the invention is read in conjunction with the accompanying drawings.

### BRIEF DESCRIPTION OF THE DRAWINGS

FIG. 1 is a diagram of the amino acid sequence of human IFN-β (SEQ ID NO: 1).

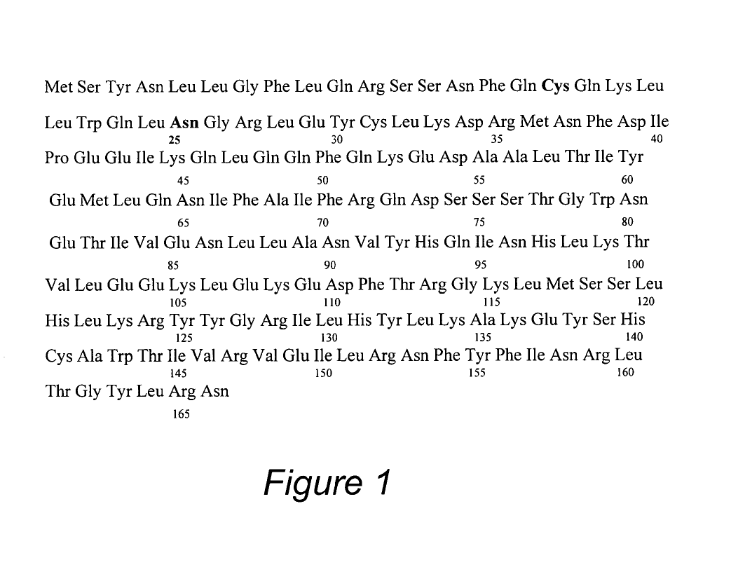

FIG. 2 is a diagram illustrating the Asp25 IFN-β degradation pathway depicting the aspartate residue at position 25, numbered in accordance with native IFN-β.

FIG. 3 is a diagram of the amino acid sequence of Asp25 IFN-β 1b (SEQ ID NO: 2) indicating the site of Asn25Asp mutation, and Lys-C cleavage sites in accordance with the present invention.

FIG. 4A shows reduced Lys-C peptide maps of Asp25 IFN-β 1b and Asn25 IFN-β 1b

FIG. 4B shows an expanded view of a portion of the map of FIG. 4A.

FIG. 5A shows reduced Lys-C peptide maps of Asp25 IFN-β 1b and Asp25 IFN-β 1b high-temperature treated sample.

FIG. 5B shows an expanded view a portion of the map of FIG. 5A.

FIG. 6 shows Mono-S cation exchange (CEX) HPLC of Asn25 IFN-β 1b, Asp25 IFN-β 1b and Asp25 IFN-β 1b high-pH treated sample.

### DESCRIPTION OF SPECIFIC EMBODIMENTS OF THE INVENTION

The compounds, compositions, materials and associated techniques and uses of the present invention will now be described with reference to several embodiments. Important properties and characteristics of the described embodiments are illustrated in the structures in the text. While the invention will be described in conjunction with these embodiments, it should be understood that the invention is not intended to be limited to these embodiments. On the contrary, it is intended to cover alternatives, modifications, and equivalents as may be included within the spirit and scope of the invention as defined by the appended claims. In the following description, numerous specific details are set forth in order to provide a thorough understanding of the present invention. The present invention may be practiced without some or all of these specific details. In other instances, well known process operations have not been described in detail in order not to unnecessarily obscure the present invention.

### INTRODUCTION

The present invention provides a recombinantly produced interferon-β protein analog which exhibits a biological activity of human interferon-β (e.g. IFN-β 1b) at an increased level relative to IFN-β 1b. The protein analog may be formulated with or without HA, but does not require HA for protein stabilization. Specifically, interferon-β protein analog contains a human interferon-β analog, in which the asparagine at position 25, numbered in accordance with native interferon-β, is recombinantly replaced by an aspartate residue. This interferon-β protein analog is free of the native Asn25 IFN-β.

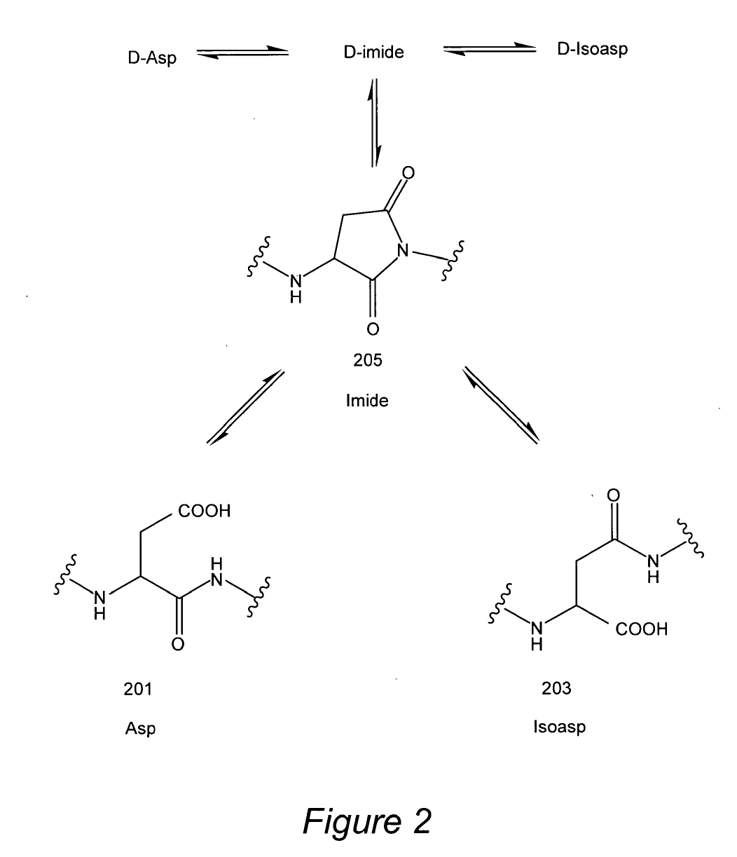

In one embodiment, the recombinantly produced interferon-β protein analog may also contain decomposition products of Asp25 IFN-β, such as products shown in FIG. 2. Referring to a scheme shown in FIG. 2 which describes the main decomposition pathway of Asp25 IFN-β, the Asp25 residue 201 can be converted to an isoaspartate residue 203 (Isoasp25) via a succinimide intermediate 205 (Imide 25). Such conversion may occur, for example, upon exposure to high-temperature or high-pH conditions. The interferon-β analogs substituted by isoaspartate or succinimide at position 25 do not significantly decrease biological activity of the recombinant Asp25 IFN-β analog and can be present in the IFN-β analog product and its therapeutic formulations. The Asn25Asp mutation is introduced using rDNA techniques, by providing a gene coding for Asp25 IFN-β, and by introducing this gene into the cells, such as microbial cells, or eukaryotic cells (e.g. CHO cells or insect cells), to effect the expression of the Asp25 IFN-β protein analog in these cells. In some embodiments, rDNA used for transformation of the cells also includes a promoter sequence.

The IFN-β protein analogs can be characterized using a reduced Lys-C endoproteinase assay performed at pH of at least 6.5, preferably at pH of about 7. Lys-C digest performed at this pH cleaves the protein at specific sites without altering the relative amounts of the decomposition products in the sample. The peptide fragments obtained in the digest can be further separated by RP HPLC, collected and identified by Edman sequencing.

Formulations of the active protein analog compounds in therapeutic compositions and methods of making and use are also provided.

A “protein analog” refers herein to a synthetic protein in which one or more amino acids has been genetically and/or chemically modified and that retains a biological activity of the parent protein, such as cellular cytopathic effects or antiproliferative activity. In a specific embodiment, the recombinant human interferon-β protein analog contains a human interferon-β 1b protein analog in which cysteine at position 17, numbered in accordance with native interferon-β, is deleted or replaced by a neutral amino acid, in particular serine, and the asparagine at position 25, is recombinantly replaced by an aspartate (e.g., IFN-β_{ser17,asp25}). In other embodiments, the Asp25 is further chemically modified to an isoaspartate or succinimide residue (e.g., IFN-β_{ser17,isoasp25} or IFN-β_{ser17,succinimide25}, respectively). The Asp25 IFN-β 1b protein analog exhibits a biological activity of human interferon-β at an increased level relative to IFN-β 1b. In addition, the enhanced biological activity is observed in an HA-free formulation of this protein analog, enabling an HA-free IFN-β 1b therapeutic.

The Replacement of Asn25 to Asp25

In the synthetic protein analog of the invention, the cysteine 17 residue may be deleted or replaced by a serine, threonine, glycine, alanine, valine, leucine, isoleucine, histidine, tyrosine, phenylalanine, tryptophan or methionine. In a specific embodiment, the substitution is serine 17. The asparagine 25 residue is replaced by an aspartate residue using

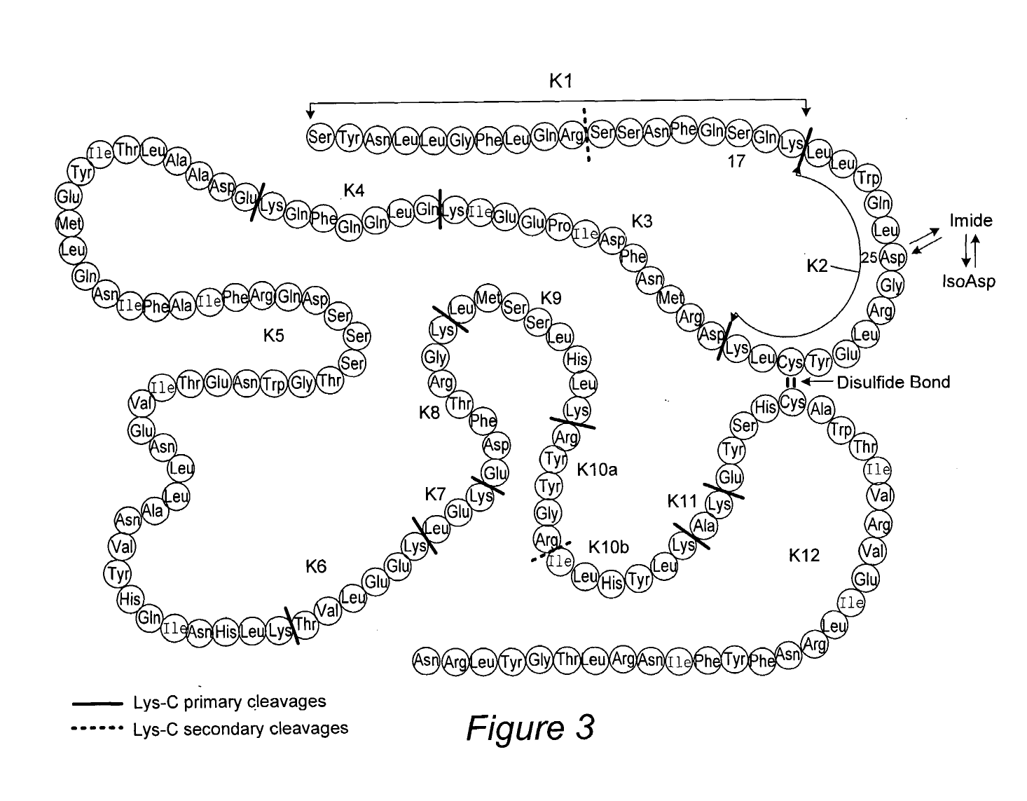

rDNA techniques. Referring to FIG. 3, the primary (amino acid sequence (SEQ ID NO: 2)) and secondary (folding, cross-linking) structure of an Asp25 IFN-β 1b protein analog in accordance with the invention is illustrated. At position 25, the native Asn residue is replaced with the recombinantly introduced Asp residue. The Asp 25 residue can be partially or substantially converted to succinimide and/or isoaspartate, for example, by exposing the Asp25 IFN-β analog to isomerisation-inducing conditions, such as high temperature or high pH. These conditions may also induce optical isomerisation of aspartate from an L form to a D form. The present invention encompasses both L and D forms of Asp25, Isoasp25, and Imide25 IFN-β protein analogs.

The protein analogs are made by recombinant synthetic techniques, which may be optionally supplemented by post-expression chemical modification. The Asp25 IFN-β 1b synthetic protein analog is prepared by recombinant DNA directed mutagenesis techniques. The Cys17Ser exchange has been described in the patents referenced above in the background section of the application. Directed mutagenesis techniques are well known and have been reviewed by Lather, R. F. and Lecoq, J. P. in Genetic Engineering Academic Press (1983) pp 31-50. Oligonucleotide-directed mutagenesis is specifically reviewed by Smith, M. and Gillam, S. in Genetic Engineering: Principles and Methods, Plenum Press (1981) 3:1-32. The Asn25Asp mutation will be described herein in further detail. It should be understood that the following mutagenesis description is provided as an example only and is not intended in any way to limit the invention. The present invention also encompasses modifications of the following protocol that will be obvious to those skilled in the art.

The mutation is introduced by means of a PCR (polymerase chain reaction) on an IFN-β producing plasmid (pSY2501) serving as a template. A Quik-Change Mutagenesis kit (Stratagene kit #200516, Stratagene, La Jolla, Calif.) can be used. Synthetic oligonucleotides are employed in PCR as primers. The primers that are used in the Asn25Asp mutagenesis are shown below:

Asp25 forward primer BSN25DF (SEQ ID NO: 3):
5′-CAGAAGCTCCTGTGGCAATTGGATGGGAGGCTTGAATATTGC-3′
Asp25 reverse primer BSN25DR (SEQ ID NO: 4):
5′-GCAATATTCAAGCCTCCCATCCAATTGCCACAGGAGCTTCTG-3′

The forward primer (SEQ ID NO: 3) contains the aspartate encoding nucleotides GAT. The reverse primer (SEQ ID NO: 4) is complementary to the forward primer. The two primers bind to different strands of plasmid DNA with opposite orientation but at the same position. The entire plasmid is replicated by PCR, and is then digested by the DpnI enzyme. DpnI digests only methylated bacterial template DNA strands while leaving the unmethylated PCR-replicated plasmids intact. The DpnI treated reaction mixture is then added to XL Gold Ultracompetent cells (Stratagene) effecting the transformation of the cells with the mutation-carrying plasmid. The cell colonies are grown in Luria agar media in the presence of carbenicillin and tryptophane. Several colonies are then picked, and are cultured in Luria media in the presence of carbenicillin and tryptophane. The plasmid DNA is extracted from each culture and is analyzed by sequencing the IFN-β insert of the plasmid. The sequencing of the IFN-β insert confirms the Asn25 to Asp25 mutation. An extensive restriction digest of the rest of the plasmid is also performed. The IFN-β insert contains a promoter directly followed by a gene encoding the Asp25 IFN-β 1b protein. The nucleotide sequences for the Asp25 IFN-β 1b encoding gene (SEQ ID NO: 5) and the IFN-β insert (gene and promoter, SEQ ID NO: 6) are provided below. The aspartate encoding codon GAT and stop-codon TGA are underlined.

Asp25 IFN-β 1b encoding gene sequence

(SEQ ID NO: 5)

ATGAGCTACAACTTGCTTGGATTCCTACAAAGAAGCAGCAATTTTCAGAG
TCAGAAGCTCCTGTGGCAATTGGATGGGAGGCTTGAATATTGCCTCAAGG
ACAGGATGAACTTTGACATCCCTGAGGAGATTAAGCAGCTGCAGCAGTTC
CAGAAGGAGGACGCCGCATTGACCATCTATGAGATGCTCCAGAACATCTT
TGCTATTTTCAGACAAGATTCATCTAGCACTGGCTGGAATGAGACTATTG
TTGAGAACCTCCTGGCTAATGTCTATCATCAGATAAACCATCTGAAGACA
GTCCTGGAAGAAAAACTGGAGAAAGAAGATTTCACCAGGGGAAAACTCAT
GAGCAGTCTGCACCTGAAAAGATATTATGGGAGGATTCTGCATTACCTGA
AGGCCAAGGAGTACAGTCACTGTGCCTGGACCATAGTCAGAGTGGAAATC
CTAAGGAACTTTTACTTCATTAACAGACTTACAGGTTACCTCCGAAACTG
AAGATCC
Promoter and gene sequence used for expression of
Asp25 IFN-β 1b

(SEQ ID NO: 6)

GAATTCCGACATCATAACGGTTCTGGCAAATATTCTGAAATGAGCTGTTG
ACAATTAATCATCGAACTAGTTAACTAGTACGCAAGTTCACGTAAAAAGG
GTATCGATAAGCTTATGAGCTACAACTTGCTTGGATTCCTACAAAGAAGC
AGCAATTTTCAGAGTCAGAAGCTCCTGTGGCAATTGGATGGGAGGCTTGA
ATATTGCCTCAAGGACAGGATGAACTTTGACATCCCTGAGGAGATTAAGC
AGCTGCAGCAGTTCCAGAAGGAGGACGCCGCATTGACCATCTATGAGATG
CTCCAGAACATCTTTGCTATTTTCAGACAAGATTCATCTAGCACTGGCTG
GAATGAGACTATTGTTGAGAACCTCCTGGCTAATGTCTATCATCAGATAA
ACCATCTGAAGACAGTCCTGGAAGAAAAACTGGAGAAAGAAGATTTCACC
AGGGGAAAACTCATGAGCAGTCTGCACCTGAAAAGATATTATGGGAGGAT
TCTGCATTACCTGAAGGCCAAGGAGTACAGTCACTGTGCCTGGACCATAG
TCAGAGTGGAAATCCTAAGGAACTTTTACTTCATTAACAGACTTACAGGT
TACCTCCGAAACTGAAGATCC

The Asp25 IFN-β was expressed in bacterial cells using standard protein expression techniques. E. Coli cells expressing tryptophane repressor were transformed with the plasmid carrying Asp25 IFN-β 1b gene. The cells were cultured to express Asp25 IFN-β 1b, and the expressed protein was isolated and purified.

Those skilled in the art will appreciate that other expression systems can be used, such as mammalian cells (HEK293 or CHO-K), baculovirus/insect cells, or yeast systems. Expression performed in some of these systems (e.g. in eukaryotic cells) may result in Asp25 IFN proteins without terminal Met deletion or in glycosylated proteins, and these are within the scope of the present invention.

The identity of the Asp25 mutant protein was confirmed by Edman sequencing of peptide fragments, which were obtained by subjecting the protein to reduced Lys-C endoproteinase mapping digest at pH 7. It was determined that the recombinantly produced Asp25 IFN-β 1b contains a major Asp25 species and two minor decomposition products, Isoasp25 and Imide25 IFN-β 1b analogs. These products are formed from the recombinant Asp25 IFN-β via a decomposition pathway illustrated in FIG. 2. The amount of Isoasp25 and Imide25 species can be altered by chemical means or by thermal treatment. For example, the fraction of Isoasp25 species can be increased when Asp25 IFN-β is subjected to high-temperature treatment. The relative proportions of Asp25, Isoasp25 and Imide25 species in the IFN-β protein analog can also be changed by varying the pH of the mixture. For instance, the relative amount of Imide25 fraction decreases at higher pH, such as 9.25. The term “human IFN-β protein analog” encompasses recombinant Asp25 IFN-β and its decomposition products such as Isoasp25 and Imide25. It should be understood that recombinant Asp25 IFN-β protein analog is completely free of Asn25 IFN-β. While it is possible to obtain interferon analogs which are deamidated at position 25 via chemical deamidation of asparagine residue, as described in the U.S. patent application Ser. No. 11/271,516, the chemically deamidated interferon products commonly contain unreacted native Asn25 protein. The Asn25 interferon exhibits lower biological activity than Asp25 IFN-β protein analog, and it is, therefore, advantageous to obtain interferon analogs which are Asn25-free.

The biological activity of recombinant Asp25 IFN-β protein analog product was explored by performing cellular cytopathic effects (CPE) assay and Hs294T antiproliferation assay. The CPE activity of Asp25 IFN-β 1b is 5.82×10⁷ IU/mg which is 1.6 times higher than CPE activity of HA-free Asn25 IFN-β 1b and 1.7 times higher than CPE activity of HA-formulated IFN-β 1b (Betaseron®). The Hs294T antiproliferation biological activity of Asp25 IFN-β 1b is 5.72×10⁷ IU/mg which is 1.3 times higher than the Hs294T antiproliferation activity of HA-free Asn25 IFN-β 1b and 1.7 times higher than corresponding activity of HA-formulated IFN-βₛₑᵣ₁₇.

The Asp25 IFN-β protein analog retains its biological activity in the absence of HA under a number of conditions, which include high pH and high temperature conditions. Therefore, formulation with HA is not required in order to maintain the biological activity of therapeutic products containing Asp25 IFN-β. Characterization of the high pH or high temperature treated HA-free Asp25 IFN-β 1b samples was carried out by RP HPLC and CEX HPLC which can be performed either on intact proteins or on peptide mixtures obtained by Lys-C digest. No unexpected peaks that may be indicative of unexpected protein degradation, were observed both in the samples subjected to high pH (e.g. pH 9.25) and in the samples subjected to high temperature (e.g. 37° C.) treatments. The presence of Asp25, Isoasp25, and Imide25 species was confirmed for all of the samples. Exposure of the samples to 37° C. for 18 days leads to increased amounts of Isoasp25 fraction in the IFN-β protein analog. Exposure of the samples to pH 9.25 for four hours results in a decrease of Imide25 species fraction, and in increase in Asp or Isoasp fractions.

It has also been observed that the biological activity of the recombinant Asp25 IFN-β protein analog IFN-β is increased when the Asp25 IFN-β is exposed to high temperature. The CPE activity of Asp25 IFN-β 1b samples incubated at 37° C. for 18 days exhibits a 2.9 fold increase relative to HA-formulated IFN-β 1b and a 2.7 fold increase relative to HA-free IFN-β 1b and reaches 9.6×10⁷ IU/mg. The increased biological activity may be attributed to higher proportions of Isoasp25 or Imide25 species in the IFN-β protein analog.

In general, there are a number of possible techniques that can lead to increased amounts of Isoasp25 or Imide25 analogs in the recombinantly produced Asp25 IFN-β protein analog. These techniques may be effective and suitable for adoption in large scale pharmaceutical manufacturing and any technique that changes the relative amounts of Asp25, Isoasp25, and Imide25 while retaining a native biological activity, preferably an increased biological activity, may be used. Broadly speaking, the relative amounts of Asp25, Isoasp25, and Imide25 IFN-β analogs can be varied by incubation at moderate to high temperature (e.g., about 25-60° C.) and a variety of pHs, from low (e.g., about 0-4) moderate (e.g., about 4-10) to high (e.g., about 10-14) with reaction times from about 1 minute to about 90 days or more, depending upon conditions. For example, IFN-β protein analog with increased biological activity may be obtained by incubation of Asp25 IFN-β (e.g., Asp25 IFN-β 1a or Asp25IFN-β 1b) for up to 60° C.; or between about 25 and 40° C., for example about 37° C., for at least 24 hours, for example 18 days or up to 40 days.

The techniques for preparing the recombinant human Asp25 IFN-β protein analog produce a product that does not contain Asn25 species. The relative amounts of Asp25, Isoasp25 and Imide25, may vary depending on conditions of recombinant protein production and subsequent chemical treatments, such as exposure to high temperature or high pH. For example, IFN-β protein analog containing at least 25%, at least 50%, at least 75% or about 100% of Asp25 IFN-β may be obtained. In some instances, it may be desirable to purify and isolate the individual species of IFN-β protein analog mixture. This purification and isolation may be achieved by cationic exchange (CEX) HPLC for example using the following conditions:
Chromatography Stationary Phase: Pharmacia Mono S HR 5/5, or equivalent;
Eluent Buffer: 20 mM Tris-HCl, pH 7.0 with 0.5% Empigen BB (n-Dodecyl-N,N-dimethylglycine (the alkyl betaine lauryldimethylbetaine, an amphoteric surfactant))
Gradient: NaCl linear gradient up to 200 mM or higher in the eluent buffer.
   For manufacturing, this technique can be conducted on a large scale.

In one embodiment, when the synthetic protein analog is microbially produced, it is unglycosylated. Also, the protein analog has an N-terminal methionine deletion. In other embodiments, the protein analog may be produced in mammalian cells and thus be glycosylated.

As described in further detail below, various activity assays demonstrate that recombinant Asp25 IFN-β 1b protein analog has increased bioactivity relative to its parent Asn25 IFN-β 1b protein. The stability results have been obtained with HA-free samples indicating that the recombinant IFN-β protein analog of the present invention is suitable for HA-free formulation as a therapeutic. To form a therapeutic composition, the protein analog, partially or substantially pure as described above, can be admixed with a pharmaceutically acceptable carrier medium, such as are well know for this type of therapeutic product.

While it is an advantageous property of compositions of the present invention that they exhibit HA-free stability, the Asp25 IFN-β protein analog may also be utilized in HA-containing formulations and these are not excluded from the scope of the present invention. In addition, while the invention is primarily described here with reference to IFN-β 1b protein analogs, the invention is also applicable to other IFN-β analogs, including IFN-β 1a analogs, and other functional variants of IFN-β protein.

The IFN-β protein analogs and compositions of the present invention exhibit biological activity that suggest utility in therapeutics in a number of applications including regulating cell growth in a patient and treating a patient for viral disease. Therapeutics in accordance with the present invention may prove to be useful in treatment of multiple sclerosis in a patient, in particular MS of relapsing-remitting type.

### EXAMPLES

The following examples illustrate aspects of the present invention, but are not intended in any way to limit the invention.

### Example 1

### Preparation of Recombinant Asp25 IFN-β 1b

Representative compositions of PCR mixtures and a representative PCR protocol for the preparation of recombinantly deamidated IFN-β 1b are illustrated in Table 1 and Table 2 respectively.

TABLE 1
Compositions of the PCR mixture

| PCR Mixture | | μL | μL |
|---|---|---|---|
| (51 μL total | Template DNA | 2 | 5 |
| volume): | (10 ng/μL) | | |
| | Forward Primer | 1.25 | 1.25 |
| | (SEQ ID NO: 3) | | |
| | (100 ng/μL) | | |
| | Reverse Primer | 1.25 | 1.25 |
| | (SEQ ID NO: 4) | | |
| | (100 ng/μL) | | |
| | Buffer 10X | 5 | 5 |
| | dNTP mixture | 1 | 1 |
| | Quick solution | 3 | 3 |
| | Water | 36.5 | 33.5 |
| | Pfu Turbo DNA | 1 | 1 |
| | Polymerase | | |

TABLE 2
Representative PCR protocol

| PCR protocol | Duration | | Temperature |
|---|---|---|---|
| Step 1 | 1 | minute | 95° C. |
| Step 2 | 50 | seconds | 95° C. |
| Step 3 | 50 | seconds | 60° C. |
| Step 4 | 1 minute/kilobase | | 68° C. |
| | | | Go to step 2. Repeat 18 cycles. |
| Step 5 | 7 | minutes | 95° C. |
| Step 6 | | | 4° C. |

Upon completion of PCR, DpnI (2 μL) was added to each reaction mixture containing tube, and the tubes were incubated for 1 hour at 37° C. The DpnI containing mixtures (2 μL) were then added to XL Gold Ultracompetent cells (45 μL of cells and 2 μL of β-mercaptoethanol). The resulting mixtures were kept at 0° C. for 30 minutes, then were kept at 42° C. for 45 seconds, and upon addition of SOC culture medium (500 μL) were incubated for 1 hour at 37° C. with 250 rpm shaking. The cells were plated in Luria-B/L-Carbenicillin (100 μg/mL) and tryptophane (50 μg/mL) plates. The colonies were allowed to grow for 24 hours at 37° C. Six colonies were picked and each was transferred in a separate tube with Luria Broth media (2 mL) containing carbenicillin (100 μg/mL) and tryptophane (50 μg/mL). The bacteria were allowed to grow for 24 hours at 37° C. The DNA was obtained from the cell cultures according to standard Qiagen protocol. The identity of DNA was determined by custom sequencing. E. coli MM294-1 cells were then transformed with the plasmid DNA containing the Asp25 IFN-β 1b insert. The cells were inoculated in a medium supplemented with tryptophane. Cells were cultured at 37° C. for approximately 21 hours and were harvested after tryptophane was depleted. The expressed Asp25 IFN-β 1b protein was isolated and purified using standard biochemical techniques.

### Example 2

### Potency Increase in Recombinant Asp25 IFN-β 1b

Results of CPE and Hs294T anti-proliferation bioactivity assays are summarized in Table 3 and Table 4 respectively. Table 3 presents CPE activity of Asp25 IFN-β 1b, heat-treated and high-pH treated Asp25 IFN-β 1b compared to CPE activity of HA-formulated (Betaseron®) and HA-free Asn25 IFN-β 1b.

TABLE 3
CPE bioactivity of IFN-β 1b protein analogs.
Specific activity (×10⁷ IU/mg)

| | HA- | | | Asp25 | |
|---|---|---|---|---|---|
| | formulated | HA-free | | IFN | Asp25 IFN |
| Assay | Betaseron ® | IFN | | 18 days | High pH |
| Day | Control | Control | Asp25 IFN | @ 37° C. | 9.25 |
| 1 | 3.32 | 3.50 | 5.09 | | |
| 2 | 3.32 | 4.38 | 6.24 | | |
| 3 | 3.26 | 3.40 | 6.03 | | |
| 4 | 3.50 | 3.67 | 6.30 | | |
| 5 | 3.40 | 3.31 | 5.46 | 9.60 | 5.96 |
| Ave (5) | 3.36 | 3.65 | 5.82 | | |
| SD | 0.09 | 0.43 | 0.53 | | |
| % RSD | 2.76 | 11.73 | 9.06 | | |

| | Fold increase | | |
|---|---|---|---|
| Betaseron Control | 1.7 | 2.9 | 1.8 |
| HA-Free Control | 1.6 | 2.6 | 1.6 |

Table 4 presents Hs294T anti-proliferative activity of Asp25 IFN-β 1b compared to corresponding activity of HA-formulated (Betaseron®) and HA-free Asn25 IFN-β 1b.

TABLE 4
Hs294T anti-proliferative activity of IFN-β 1b protein analogs.
Specific activity (×10⁷ IU/mg)

| | HA-formulated | | |
|---|---|---|---|
| Assay | Betaseron ® | HA-free IFN | |
| Day | Control | Control | Asp M-IFN |
| 1 | 3.12 | 4.05 | 4.92 |
| 2 | 3.43 | 4.93 | 5.66 |
| 3 | 3.08 | 4.17 | 6.33 |
| 4 | 3.02 | 3.67 | 4.86 |
| 5 | 3.93 | 4.40 | 6.80 |
| Ave (5) | 3.32 | 4.24 | 5.72 |
| SD | 0.38 | 0.47 | 0.85 |
| % RSD | 11.40 | 11.01 | 14.91 |

| | Fold Increase |
|---|---|
| Betaseron Control | 1.7 |
| HA-Free Control | 1.3 |

### 2.1. CPE Bioassay

IFN-β induces an antiviral state in mammalian cells in which some virus types are inhibited from replicating and causing cellular cytopathic effects (CPE). A549 human lung carcinoma cells and murine encephalomyocarditis (EMC) virus were used to evaluate biological activity of the Asp25 IFN-β 1b and its high temperature (about 18 days at 37° C.) and high pH (pH 9.25 for 4 hours) treated samples.

Serial dilutions of the IFN-β test samples were performed in 96-well plates. A549 cells were prepared in tissue culture medium and added to the assay plates. After incubation overnight, the EMC virus is added to the assay plates followed by an additional overnight incubation to allow virus replication. Cells treated with sufficient IFN-β 1b were protected from the virus challenge and remained viable. Unprotected cells underwent cytopathic changes and died. The interferon dose dependent CPE was quantitated using a phosphatase (pNPP) staining technique and a dose response curve was prepared from a plot of cell viability (Optical Density measurement) vs. IFN-β concentration. IFN-β activity was calculated from the ratio of the ED₅₀ (the concentration required for half maximal cell protection) of the test sample and a WHO calibrated reference standard.

As shown in Table 3, the CPE activity of HA-free Asp25 IFN-β 1b is significantly higher than that of the HA-formulated IFN-β 1b (1.7 fold increase) and the HA-free Asn 25 IFN-β 1b (1.6 fold). The CPE activity of heat-treated HA-free Asp25 IFN-β 1b is even higher (2.9 fold increase relative to HA-formulated IFN-β 1b and 2.6 fold relative to the HA-free Asn 25 IFN-β 1b). High-pH treated Asp25 IFN-β 1b preserves its high activity which is 1.8 and 1.6 times higher than the CPE activity of IFN-β 1b and HA-free Asn 25 IFN-β 1b respectively.

### 2.2 Antiproliferative Activity

IFN-β shows antiproliferative activity against a number of cell lines established from human tumors. A human melanoma cell line (Hs294T) was used to evaluate biological activity of the HA-free Asn25 IFN-β 1b and HA-free Asp25 IFN-β 1b samples

Serial dilutions of the IFN-β test samples were performed in 96-well plates. Responder cells were prepared in tissue culture medium and added to the assay plates. After 3 days of incubation, the cells were stained with pNPP (a phosphatase stain) to measure the growth response. Cell growth was inhibited in response to IFN-β in a dose dependent manner. A dose response curve was prepared from a plot of cell number (Optical Density measurement) vs. IFN-β concentration. IFN-β activity was calculated from the ratio of the ED₅₀ (the concentration required for half maximal cell growth response) of the test sample and a WHO calibrated reference standard.

As shown in Table 4, the antiproliferative activity in the Asp25 IFN-β 1b is significantly higher than that in the HA-formulated IFN-β 1b (1.7 fold increase) and the HA-free Asn 25 IFN-β 1b (1.3 fold).

### Example 3

### Characterization of the Recombinant Asp25 IFN-β 1b

Recombinant Asp25 IFN-β 1b has the asparagine residue in position 25, numbered in accordance with native interferon, recombinantly replaced by an aspartate residue. It also carries the Cys17Ser mutation. The primary sequence of Asp25 IFN-β 1b is shown in FIG. 3. The aspartate at position 25 can be converted to succinimide and isoaspartate species according to a degradation pathway illustrated in FIG. 2. Recombinant IFN-β 1b protein analog of the present invention encompasses the Asp25 mutant protein as well as Imide25 and Isoasp25 interferons derived from the Asp25 mutant. The composition of recombinant IFN-β 1b protein analog of the present invention was explored in the assays described below:

### 3.1. Reduced Lys-C Peptide Map

A peptide map produces a fingerprint profile for proteins using an enzymatic digest followed by RP-HPLC. Each of the peptide fragments separated by RP-HPLC, may be isolated and further characterized by other analytical methods, such as mass spectrometry or Edman peptide sequencing. Peptide mapping is commonly utilized in quality control as an ID test. It is also a powerful tool to monitor minor primary structure modifications in a protein from events such as clipping, mutation and degradation due to oxidation or deamidation. Chemical deamidation studies, described in the U.S. patent application Ser. No. 11/271,516, showed that chemically deamidated IFN-β contains aspartate, succinimide, and isoaspartate variants, wherein the deamidation occurs only at position 25. The peptide mapping that is used for analyzing recombinant Asp25 IFN-β protein analog, should, therefore, be carried out under conditions that do not alter the distribution of Asp25, Imide25, and Isoasp25 species in the sample. It is known that at high pH the cyclic imide is unstable, and is artificially converted to other species. In one aspect, the present invention provides a unique endoproteinase digest that is performed at essentially neutral pH, at least at about 6.5, preferably at a pH from about 6.9 to about 7.1, and more preferably at pH of about 7. This digest does not artificially induce the decomposition of succinimide and, therefore, does not affect the distribution of Asp25, Imide25 and Isoasp25 species in the analyzed sample. This digest can be carried out in the presence of solubilizing agents, such as Empigen BB, an alkyl betaine amphoteric surfactant, in those cases when the protein lacks solubility in the indicated pH range. These solubilizing agents may also include nonionic (e.g. Tween 80, available from Sigma-Aldrich, Milwaukee Wis.), cationic, anionic or amphoteric surfactants, known to those skilled in the art to be compatible with Lys-C endoproteinase. Protein mapping with this digest is particularly suitable for analysis of IFN-β but can also be applied for analysis of other proteins, such as proteins which are unstable at high pH.

A new reduced lysyl endoproteinase(R)(Lys-C) peptide map has been developed coupled with a reducing agent functional at a pH of at least about 6.5, preferably at about 6.9-7.1, to characterize the species present in the recombinant IFN-β protein analog A suitable reducing agent which can be used in this assay is dithiothreitol (DTT). The Lys-C digest of recombinant IFN-β 1b protein analog was performed using the sample preparation including digestion at pH of about 7.0 and subsequent reduction, which is performed at the optimal pH range of about 6.9-7.1. This range is needed to both preserve the native level of Asp25, Isoasp25, and Imide25 forms, in the sample and to effectively reduce and cleave the protein. Since the peptide map developed here employs a neutral pH sample preparation, the accurate monitoring of the native level of Asp25 IFN-β and its degradation products is successfully achieved. Other suitable reducing agents in conjunction with which the method can be used may include tris-(2-carboxyethyl) phosphine (TCEP), 2-mercaptoethanol, cysteine, reduced glutathione, 2-mercaptoethylamine and thioglycollic acid.

Samples of recombinant Asp25 IFN-β 1b were tested by a reduced Lys-C peptide map to confirm the Asn to Asp mutation at position 25. To each 0.4 ml of IFN sample at a concentration of 0.25 to 0.5 mg/ml in 2 mM Aspartic Acid formulation buffer was added 250 μl 1M TRIS HCl at pH 7.0, 5 μl of 30% Empigen BB (available from Calbiochem, San Diego, Calif.) and 620 μl 0.01N HCl. The digest was started with the addition of 4 μl of 1 mg/ml Lys-C and incubation at 37° C. for 4 hours. Another 4 pt of 1 mg/ml Lys-C was then added and the samples were incubated at 37° C. for a total of 24 hours. To quench the digest 100 μl of 8 M guanidine was added. The digested samples were then reduced by adding 8 μl of 1M DTT followed by incubation at 37° C. for 45 minutes. The peptide fragments were resolved by RP-HPLC chromatography (Vydac 218TP54 C18, 250×4.6 mm), using an acetonitrile gradient in 0.1% trifluoroacetic acid as elution buffer, at a flow rate of 2 ml/min and a column temperature of 40° C. The Lys-C endoproteinase cleaves the interferon protein in specific sites leading to 12 major peptide fragments K1-K12. The primary and secondary Lys-C cleavage sites and the corresponding peptide fragments are illustrated in FIG. 3. The K2 fragment contains the mutated residue at position 25. Depending on the nature of this residue, the K2 fragment elutes at different retention times on RP-HPLC, allowing differentiation between Asp25, Isoasp25, and Imide 25 species. This method also allows to distinguish deamidated species from the native Asn25 IFN.

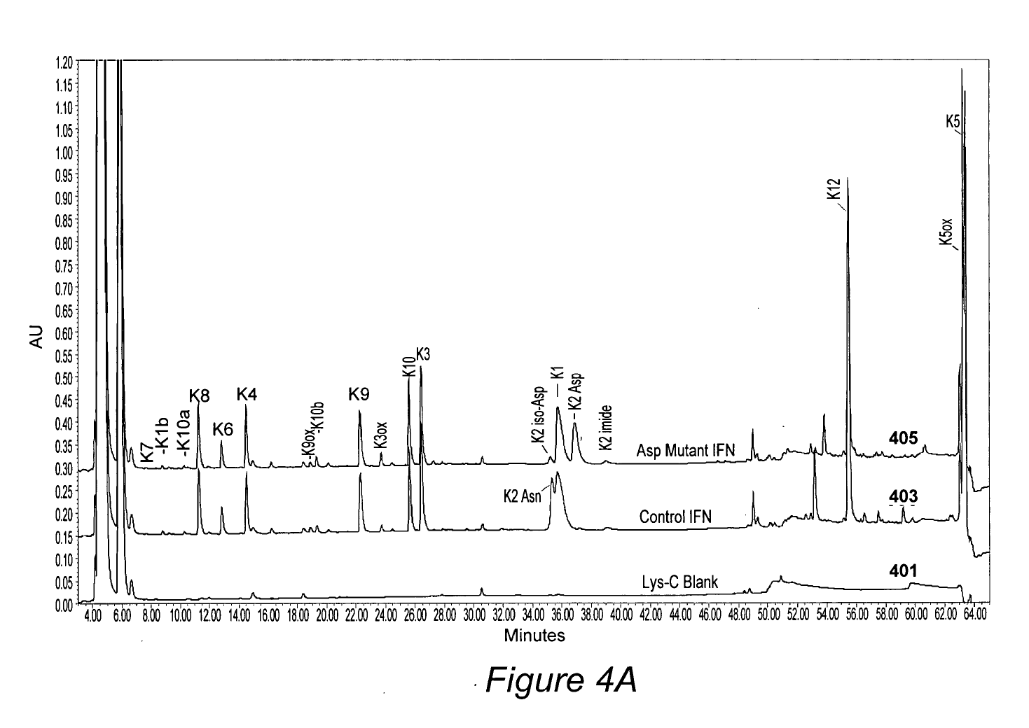

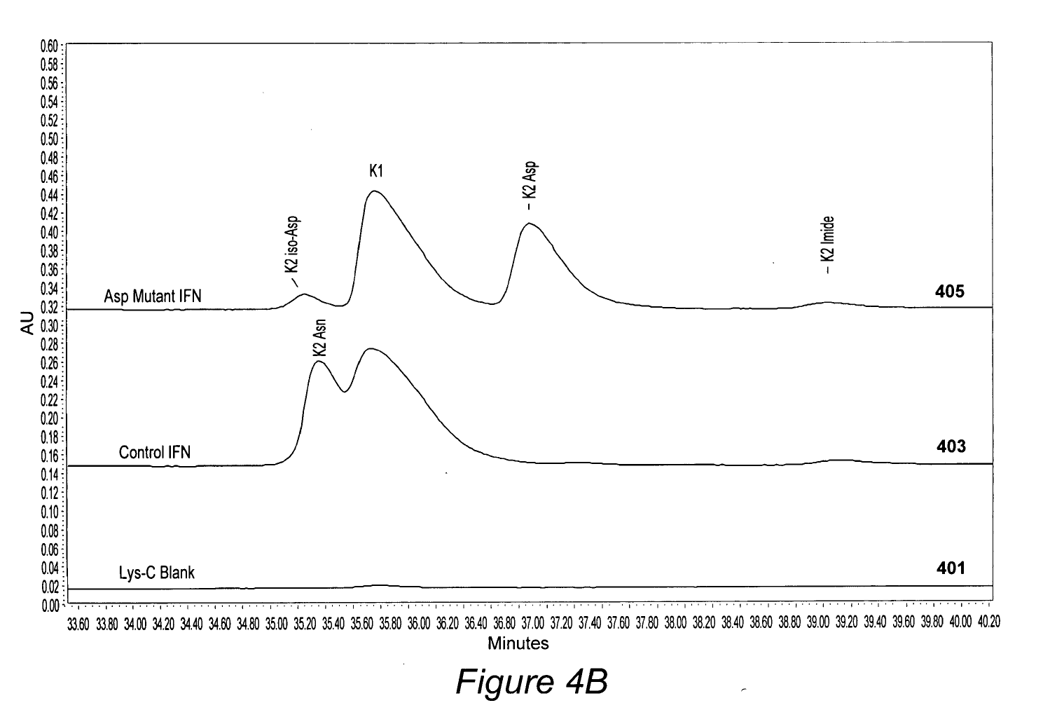

FIG. 4A shows RP-HPLC traces of Lys-C digested interferons. Trace 405 corresponds to recombinant Asp25 IFN-β 1b protein analog, trace 403 corresponds to control Asn25 IFN-β 1b and trace 401 represents the blank Lys-C enzyme sample. Peaks corresponding to K1 and K2 fragments appear in the 32-40 minute retention time interval. The expanded view of this interval is shown in FIG. 4B. It can be seen that the major K2 fragment of recombinant Asp25 IFN-β 1b protein analog elutes at a later retention time than K2 fragment of the native Asn25 IFN-β 1b control. This major fragment in the recombinantly produced Asp25 IFN-β was isolated and identified as Asp25 IFN-β 1b by mass spectrometry and Edman sequencing techniques. Two minor fragments present in the recombinant Asp25 IFN-β 1b protein analog product were identified as Imide25 and Isoasp25 species.

Edman sequencing data, confirming the identity of K1 and K2 fragments is presented in Table 5.

TABLE 5
Identification of Lys-C peptide map fragments by Edman sequencing

| Sample | Sequence | Comments |
|---|---|---|
| Recombinant Asp25 | | |
| IFN-β 1b | | |
| K1 | SYNLLGFLQRSSNFQSQK | Confirms presence of |
| | (SEQ ID NO: 7) | Ser17 |
| | | and N-terminal Met |
| | | deletion |
| K2 (major peak) | LLWQLDGR | Confirms Asp25 mutation |
| | (SEQ ID NO: 8) | |
| K2 (minor peak) | LLWQLXXX | X corresponds to no call |
| | (SEQ ID NO: 9) | at this cycle. No call at |
| | | this cycle and cycles after |
| | | it suggest that 25 Isoasp |
| | | is present |
| Control Asn25 | | |
| IFN-β 1b | | |
| K1 | SYNLLGFLQRSSNFQSQK | Confirms presence of |
| | (SEQ ID NO: 10) | Ser17 |
| | | and N-terminal Met |
| | | deletion |
| K2 | LLWQLNGR | Confirms presence of |
| | (SEQ ID NO: 11) | Asn25 |

### Example 4

### Stability of HA-Free Recombinant Asp25 IFN-β 1b Product

Since it is desirable to use recombinant Asp25 IFN-β in HA-free therapeutic formulations, stability in terms of biological activity of HA-free Asp25 IFN-β 1b was investigated. Two types of stability studies were performed. Stability to high temperature and stability to high pH was explored by subjecting HA-free Asp25 IFN-β 1b to 37° C. for 22 days and in a separate experiment to pH 9.25 for four hours.

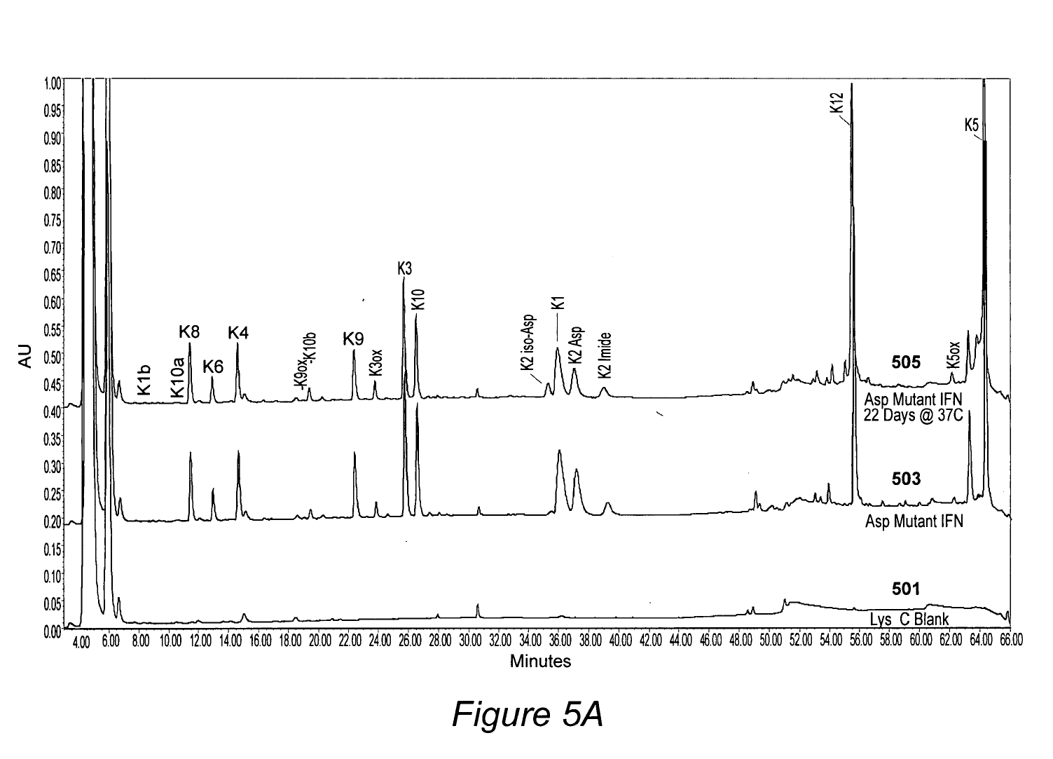

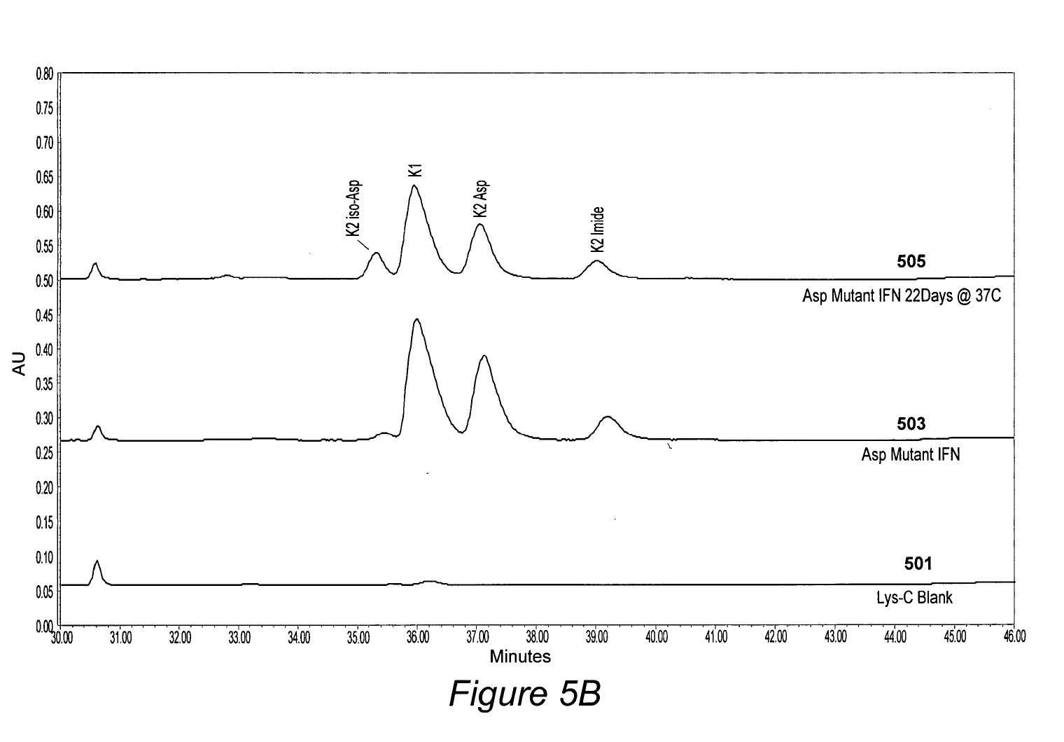

FIG. 5A presents RP HPLC data of Lys-C digested HA-free Asp25 IFN-β 1b before and after it was heat treated. Trace 501 is the trace for blank Lys-C enzyme, trace 503 corresponds to Asp25 IFN-β 1b, and trace 505 corresponds to Asp25 IFN-β 1b incubated at 37° C. for 22 days. As it can be seen from this figure, no unexpected peaks indicative of other decomposition species beyond Isoasp25 and Imide25 IFN-β analogs are observed in the high-temperature stability sample. The peaks of interest corresponding to K1 and K2 fragments are shown in greater detail in FIG. 51. It can be seen that relative amounts of Asp25, Isoasp25, and Imide25 species change upon heat treatment. Notably, the proportion of Isoasp25 fragment is increased. This experiment demonstrates that HA-free Asp25 IFN-β 1b can withstand high-temperature exposure for prolonged periods without noticeable increase in decomposition species that adversely affect its biological activity and is, therefore, suitable for HA-free formulations.

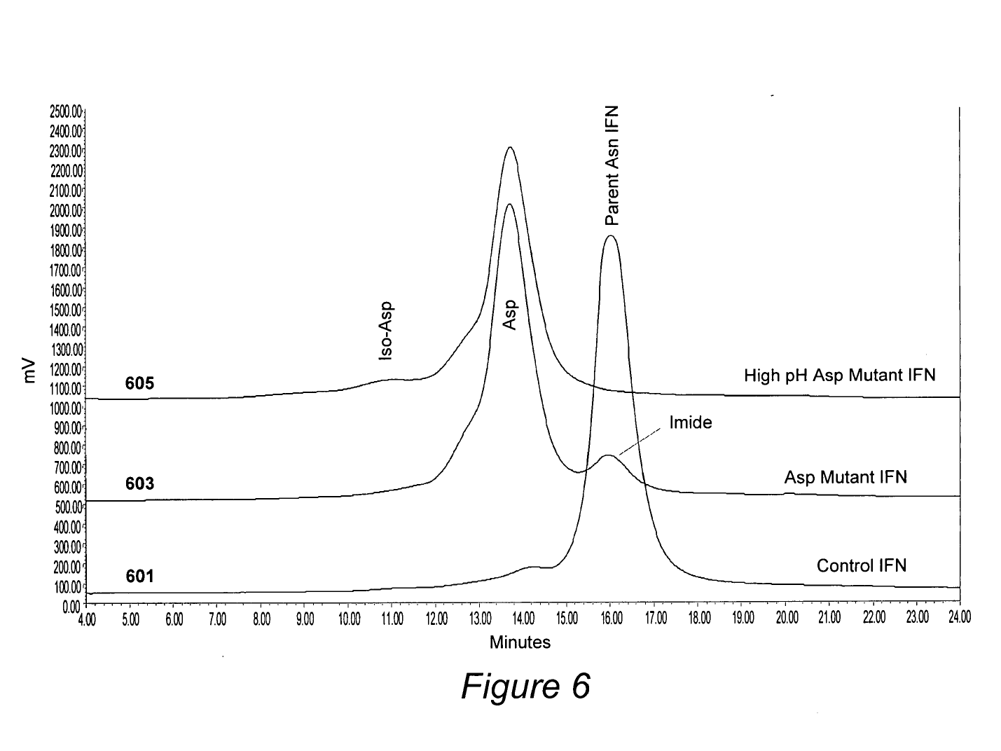

The stability of HA-free Asp25 IFN-β 1b samples at high pH was analyzed by Mono-S CEX HPLC performed on intact proteins. The chromatograms of Asp25 IFN-β 1b before and after high-pH treatment are shown in FIG. 6. No peaks indicative of unexpected degradation are observed for HA-free high-pH stability samples. Trace 601 corresponds to Asn25 IFN-β 1b, trace 603 corresponds to Asp25 IFN-β 1b, and trace 605 corresponds to Asp25 IFN-β 1b exposed to pH 9.25 for four hours. It can be seen that relative amounts of Asp25, Isoasp25, and Imide25 species change upon high-pH treatment. Specifically, Imide25 fragment is significantly reduced, while Isoasp25 species is increased. This experiment demonstrates that HA-free Asp25 IFN-β 1b does not form unexpected degradation products at high pH. Formulation with HA involves steps which are typically performed at high pH, and it is, therefore, advantageous that Asp25 IFN-β 1b retains its biological activity under high-pH conditions, and can be formulated in HA-containing therapeutic products.

It is concluded that Asp25 IFN-β 1b maintains its biological activity and does not form any unexpected degradation products both under high temperature and under high-pH conditions. It is concluded that Asp25 IFN-β 1b can be successfully formulated in HA-free or HA-containing therapeutics for treatment of multiple sclerosis and other diseases.

### CONCLUSION

Based on the results obtained from studies described above, recombinant Asp25 IFN-β protein analog (including Asp25, Isoasp25 and Imide25 IFN-β species) exhibits biological activity of IFN-β (e.g. IFN-β 1b) at an increased level. Recombinant Asp25 IFN-β, e.g., Asp25 IFN-β 1b, can be prepared in a pharmaceutically acceptable manner and can be formulated into therapeutic products with increased biological activity. The recombinant IFN-β protein analog of this invention can be prepared by introducing an Asp25 mutation into human IFN-β using site-directed mutagenesis techniques. The recombinant IFN-β protein analog of this invention may reduce the required clinical dose in HA-free and HA-containing IFN-β formulations. By lowering the clinical dose, the proportion of patients experiencing an adverse immune reaction (e.g., neutralizing antibodies), is reduced.

Although the foregoing invention has been described in some detail for purposes of clarity of understanding, it will be apparent that certain changes and modifications may be practiced within the scope of the appended claims. It should be noted that there are many alternative ways of implementing both the processes and compositions of the present invention. Accordingly, the present embodiments are to be considered as illustrative and not restrictive, and the invention is not to be limited to the details given herein, but may be modified within the scope and equivalents of the appended claims.

All documents cited herein are hereby incorporated by reference herein in their entirety and for all purposes.

What is claimed is:

1. A human interferon-β protein analog, comprising:
an Asn25-free Asp25 human interferon-β.

2. The protein analog of claim 1, wherein said protein analog exhibits a biological activity of native human interferon-β.

3. The protein analog of claim 1, comprising the amino acid sequence as set forth in (SEQ ID NO: 2).

4. The protein analog of claim 1, wherein the cysteine at position 17, numbered in accordance with native interferon-β, is deleted or replaced by a neutral amino acid.

5. The protein analog of claim 4, wherein said cysteine residue has been replaced by a serine residue.

6. The protein analog of claim 5 further comprising:
human interferon-β wherein a residue at position 25 is selected from the group consisting of isoaspartate and cyclic imide.

7. The protein analog of claim 5, wherein the protein analog is unglycosylated.

8. The protein analog of claim 7, wherein the protein analog has an N-terminal methionine deletion.

9. The synthetic protein analog of claim 2, wherein the protein analog has a biological activity greater than IFN-β 1b.

10. The protein analog of claim 9, wherein the protein analog has a biological activity at least about 1.6 times greater than HA-free IFN-β 1b.

11. The protein analog of claim 9, wherein the protein analog has a biological activity at least about 1.7 times greater than HA-formulated IFN-ii 1b.

12. A therapeutic composition having IFN-β activity comprising a therapeutically effective amount of the protein analog of claim 1 admixed with a pharmaceutically acceptable carrier medium.

13. The composition of claim 12, wherein the composition is HA-free.

14. The composition of claim 12, wherein the composition is formulated with HA.

15. A method of making an IFN-β protein analog, comprising:
transforming a cell with an Asp25 interferon-β coding sequence; and
culturing the transformed cells to express Asp25 IFN-β protein analog.

16. The method of claim 15 further comprising:
isolating and purifying said Asp25 IFN-β protein analog.

17. An Asn-free Asp25 human interferon-β protein analog prepared according to the method of claim 15.

18. A method of treating a patient comprising administering to said patient an effective amount of the composition of claim 12.

19. The method of claim 18, wherein the treatment is for multiple sclerosis in the patient and the effective amount is a therapeutically effective amount of the composition.

20. The method of claim 19, wherein said multiple sclerosis is relapsing remitting type.

21. A peptide mapping method, comprising:
incubating a protein sample in a buffered solution at pH of at least 6.5 containing a Lys endoproteinase-C;
allowing the Lys endoproteinase-C to digest the protein sample;
reducing the digested protein sample with a reducing agent; and
resolving the peptide fragments of the digested protein sample by liquid chromatography.

22. The method of claim 21, wherein the reducing agent is dithiothreitol (DTT).

23. The method of claim 22, wherein the protein sample is a human IFN-β protein analog.

24. The method of claim 21, wherein the liquid chromatography is RP-HPLC.

25. The method of claim 23, wherein the incubation is conducted at a pH of about 7.

26. The method of claim 21, wherein the buffered solution also comprises a solubilizing agent.

27. The method of claim 26, wherein the solubilizing agent is an alkyl betaine amphoteric surfactant.

28. A microorganism having a DNA sequence coding for Asp25 human interferon-β.

29. A DNA sequence coding for the Asp25 human interferon-β and comprising the sequence as set forth in (SEQ ID NO: 5).

30. The sequence of claim 29, further comprising a promoter sequence used in the expression of Asp25 human interferon-β, the coding and promoter sequences comprising the sequence as set forth in (SEQ ID NO: 6).

31. The human interferon-β analog of claim 1 having a purity acceptable for pharmaceutical utilization.

32. The method of claim 15, wherein the transformed cell is selected from the group consisting of a microbial cell, a CHO-cell, and an insect cell.

33. The method of claim 32, wherein the transformed cell is an E. coli cell.

34. The method of claim 15, wherein the transformed cell is a eukaryotic cell.


SEQUENCE LISTING (11 sequences)

SEQ ID NO: 1 - protein, 166 aa, Homo sapiens
MSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 2 - protein, 165 aa, Artificial
  Recombinant mutant Asp25 IFN-beta 1b
SYNLLGFLQRSSNFQSQKLLWQLDGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 3 - DNA, 41 bp, Artificial
  Forward primer for Homo Sapiens IFN-beta 1b
cagaagctcctgtggcaattggatgggaggcttgaatattg

SEQ ID NO: 4 - DNA, 42 bp, Artificial
  Reverse primer for Homo Sapiens IFN-beta 1b
gcaatattcaagcctcccatccaattgccacaggagcttctg

SEQ ID NO: 5 - DNA, 507 bp, Artificial
  Gene coding for Homo sapiens Asp25 IFN-beta 1b
atgagctacaacttgcttggattcctacaaagaagcagcaattttcagagtcagaagctcctgtggcaattggatgggaggcttgaatattgcctcaaggacaggatgaactttgacatccctgaggagattaagcagctgcagcagttccagaaggaggacgccgcattgaccatctatgagatgctccagaacatctttgctattttcagacaagattcatctagcactggctggaatgagactattgttgagaacctcctggctaatgtctatcatcagataaaccatctgaagacagtcctggaagaaaaactggagaaagaagatttcaccaggggaaaactcatgagcagtctgcacctgaaaagatattatgggaggattctgcattacctgaaggccaaggagtacagtcactgtgcctggaccatagtcagagtggaaatcctaaggaacttttacttcattaacagacttacaggttacctccgaaactgaagatcc

SEQ ID NO: 6 - DNA, 621 bp, Artificial
  Promoter and gene coding for Homo sapiens Asp25 IFN-beta 1b
gaattccgacatcataacggttctggcaaatattctgaaatgagctgttgacaattaatcatcgaactagttaactagtacgcaagttcacgtaaaaagggtatcgataagcttatgagctacaacttgcttggattcctacaaagaagcagcaattttcagagtcagaagctcctgtggcaattggatgggaggcttgaatattgcctcaaggacaggatgaactttgacatccctgaggagattaagcagctgcagcagttccagaaggaggacgccgcattgaccatctatgagatgctccagaacatctttgctattttcagacaagattcatctagcactggctggaatgagactattgttgagaacctcctggctaatgtctatcatcagataaaccatctgaagacagtcctggaagaaaaactggagaaagaagatttcaccaggggaaaactcatgagcagtctgcacctgaaaagatattatgggaggattctgcattacctgaaggccaaggagtacagtcactgtgcctggaccatagtcagagtggaaatcctaaggaacttttacttcattaacagacttacaggttacctccgaaactgaagatcc

SEQ ID NO: 7 - protein, 18 aa, Artificial
  Peptide fragment from a Lys-C digest
SYNLLGFLQRSSNFQSQK

SEQ ID NO: 8 - protein, 8 aa, Artificial
  Peptide fragment from a Lys-C digest
LLWQLDGR

SEQ ID NO: 9 - protein, 8 aa, Artificial
  Peptide fragment from a Lys-C digest
LLWQLXXX

SEQ ID NO: 10 - protein, 18 aa, Artificial
  Peptide fragment from a Lys-C digest
SYNLLGFLQRSSNFQSQK

SEQ ID NO: 11 - protein, 8 aa, Artificial
  Peptide fragment from a Lys-C digest
LLWQLNGR